(19) United States
(12) Patent Application Publication
(10) Pub. No.: US 2025/0090647 A1
(43) Pub. Date: Mar. 20, 2025

(54) OPTIMIZED MULTIDIMENSIONAL BIOLOGICAL ACTIVITY OF POLY-ICLC WITH CONTROLLED COMPONENT SIZE AND FORMULATION

(71) Applicant: Oncovir, Inc., Stevenson, MD (US)

(72) Inventors: Andres M. Salazar, Winchester, VA (US); Esteban Celis, Winchester, VA (US)

(21) Appl. No.: 18/445,640

(22) PCT Filed: Aug. 4, 2022
(86) PCT No.: PCT/US2022/000015

§ 371 Date: Dec. 1, 2023

Related U.S. Application Data
(60) Provisional application No. 63/259,660, filed on Jul. 30, 2021.

(51) Int. Cl. A61K 39/12; A61K 39/00; A61K 39/39; A61P 31/14; A61P 35/00

(52) U.S. Cl. CPC A61K 39/12; A61K 39/39; A61P 31/14; A61P 35/00; A61K 2039/543; A61K 2039/55555; A61K 2039/585

(57) ABSTRACT

Poly-ICLC molecules and methods for producing them, including certain specific molecular weight molecules having improved activity in certain applications. These molecules may be incorporated in pharmaceutically or veterinary acceptable excipients and carriers for a number of uses in humans, in domestic animals and in wild animals. Such uses include (but are not limited to) prophylaxis pre-exposure prophylaxis, treatment and/or inflammatory symptom attenuation of viral or microbial infections; immunomodulating, vaccine adjuvant, antiviral, and/or anti-inflammatory effects mediated through activation of the MDA5, TLR3 and other dsRNA dependent enzyme systems; antineoplastic effects either alone or when combined with therapeutic vaccine or other anticancer immunologic agents; preventive cancer vaccine adjuvant effects in patients at risk for cancer. The molecules may be of particular use against SARS-CoV-2 infection or a cytokine storm caused by a SARS-CoV-2 infection. While dosage may be adjusted depending on the specific target and the specific patient, the dose should be sufficient to activate the MDA5 and TLR3 enzyme systems in the patient. For humans the dose is between approximately 0.5 and 50 micrograms per kilogram body weight. The nature of the molecule does not demand any specific route of administration. Therefore, the route can be selected based on the specific target and the specific patient, and could include (but not be limited to) IM, SC, IT, IV or IN. Preferably, administration would comprise two or three repeated dose cycles spaced 24 to 96 hours apart. Depending on the target and the patient's response, dose cycles may be repeated 2 to 4 times per month.

### PRIORITY

This application claims priority from U.S. Provisional Patent Application 63/259,660, filed 30 Jul. 2021.

### FIELD AND BACKGROUND OF THE INVENTION

The present invention relates in general to double stranded polyribonucleotides, and more specifically to optimization of the multidimensional biological activity of Poly-ICLC by controlling its formulation and the size of its components Novel Poly-ICLC molecules having optimized biological activity are disclosed herein, as well as methods of producing such molecules, compositions, methods of treatment and medical uses thereof.

The novel Poly-ICLC molecules disclosed herein have optimized antitumor, antiviral, anti-inflammatory and adjuvant effects, and are useful for treating a wide range of diseases and conditions.
Prior Patents: (U.S. Pat. No. 4,349,538 (Levy), Worldwide Patent WO2005102278A1 (Salazar), Europe U.S. Pat. No. 12,823,628.8, PCT/US12/51161 (Bahjat), and PCT application 63/103,487_(Michels & Pearson) are incorporated herein by reference.

### BACKGROUND

A particular family of double stranded polyribonucleotides (dsRNA) of interest are the poly-ICs, which can be formed by combining polyinosinic acid (poly-1) and poly-cytidilic acid (poly-C) strands. These duplexes can be further stabilized and their biological activity markedly enhanced by compounding with poly-lysine and carboxymethyl cellulose, generating the overall complex poly-ICLC. Methods of preparation and clinical use of poly-ICLC were initially described in U.S. Pat. No. 4,349,538 (Levy '82) and further described in international publication WO2005102278A1 (Salazar). When properly administered to patients, Poly-ICLC functions essentially as a viral mimic or immune ‘danger signal’; it does not carry a specific genetic message, nor does it replicate. (Caskey, et al 2011) However, when administered IM, IV, SC, IT or nasally it generates a multidimensional innate and adaptive immune host defense, as detailed below.

As previously reported, the size of the poly-IC duplex as well as the size of the poly-I-lysine have important differential biological effects (Levy '82; Morahan PNAS '72, Kato et al 2008). Although biological systems can respond similarly to variations in certain large molecules such as poly-ICLC, relative uniformity of biological effects remains an important drug attribute for large scale production and commercialization of a specific drug product. As importantly and as disclosed here, although the role of dsRNA size has been described, the size as well as the formulation of the homopolymers used to compound the optimal poly-IC duplexes in poly-ICLC can also have a profound impact on activity and has not been detailed biologically or applied clinically in the past with regard to MDA5 activation.

Thus, not all poly-ICLCs are the same. (Levy '82) Plain poly-IC and various derivatives are generally considered to exert their immunostimulatory actions through the pattern recognition receptor (PRR) toll-like receptor 3 (TLR3), with induction of IFNb and activation of dendritic cells. However, more recent studies have demonstrated additional unique multidimensional actions of poly-ICLC (aka Hiltonol®) that are mediated largely through other PRRs, most notably the cytoplasmic dsRNA dependent Melanoma differentiation-associated protein 5 helicase (MDA5). The polylysine component in Poly-ICLC acts as a transfection agent, bursting the endosome through a proton sponge effect and delivering the drug into the cytoplasm, where it then preferentially activates MDA5. (Sultan, Celis et al, 2018). The various actions address multiple steps in the human immune response to infection and cancer. The relatively unique additional effects induced through the MDA5 system include marked expansion of CD8 T cells (including cytotoxic lymphocytes (CTL) and T memory cells) via IL-15 and other cytokines; targeting and infiltration of those CD 8 ‘killer’ T cells into tumor through a direct effect on tumor endothelium and induction of various chemokines (e.g. CXCL-10), as well as a marked reduction in myeloid derived suppressor cells (MDSC). Sultan, Salazar, Celis, 2020). In other words, these steps reflect dimensions of a systematic host response to cancer and other challenges. As a viral mimic and 20 multidimensional host response modifier, the Poly-ICLC described here can activate each of these steps when properly administered before, during and following an immune prime against the offending pathogen, for example as described in the clinical example under embodiments below.

Additionally, Poly-ICLC has been shown to have an anti-inflammatory effect, also mediated through the MDA5 system, that markedly protects against ischemic stroke by countering the cytokines and other DAMPS (damage associated molecular patterns) generated by tissue injury. As with the Acute Respiratory Distress Syndrome (ARDS) or ‘cytokine storm’ in COVID-19, this excessive inflammatory response is usually responsible for most of the ultimate morbidity and mortality following stroke and trauma. (Gesuete et al 2016). When properly dosed, Poly-ICLC can thus counter a variety of aberrant pathological responses to injury as well as help reverse infectious or neoplastic pathogen evasions in the course of generating an effective sequential immune response to that pathogen. Given the importance of MDA5 activation at the center of these multiple actions, optimization of poly-ICLC activation of this pathway has major clinical significance.

Previous clinical studies have confirmed that poly-ICLC will reliably induce several hundred innate immune related genes in a pattern that mimics that of an attenuated live virus vaccine in humans. In other words, as a reliable and authentic viral mimic, poly-ICLC is activating a broad variety of natural host defenses against a variety of pathogens as described above. (Caskey, et al 2011)

Clinical administration of Poly-ICLC can thus result in multiple beneficial effects, including interferon induction, broad immune enhancement, and modulation or activation of hundreds of genes representing multiple immune systems. Due to these effects, poly-ICLC has been broadly considered as a standalone antitumor agent, an immune modulator, an antiviral, an antiinflammatory, neuroprotective, and tissue protective agent, as well as a vaccine PAMP-adjuvant (Sultan, Salazar, Celis review September 2020) Taken together these actions can form the basis a relatively comprehensive response to viral infections, injury, and cancer. Our preclinical work in mice has indicated that MDAS is a critical pathway in anti-cancer and antiviral immune and tissue protective responses, since MDA5-deficient mice did not respond well to Poly-ICLC whereas TLR-3 deficient mice responded well. (Sultan, Celis et al 2020). (Gesuete et al. 2016)

We have developed a HEK IFN-1 reporter cell-based bioassay that primarily reflects MDA5 activity and thus the critical additional actions of poly-ICLC vs other dsRNAs, including plain Poly-IC. Certain Poly-ICLCs are capable of stimulating both MDA5 and RIG-I preferentially over TLR3 in these particular cells. We disclose here marked differences in activation of the MDAS pathway by different formulations of Poly-ICLC, and thus a method for optimizing this critical response.

The size of the poly-I and poly-C components of poly-IC has been correlated to efficiency of interferon induction and antiviral action (Morahan '72, Levy 1982). Because of the complex and inter-related clinical actions of Poly-ICLC, the overall correlation between polyribonucleotide component size and each facet of biological activity is not completely understood. Despite lacking complete understanding, it is clear that precise control of the size of the polyribonucleotide components is a highly desirable element of any production method. Targeting specific molecular weight ranges may enhance activity toward a certain indication or reduce toxicity at effective dose levels. To date, however, a reliable method of producing Poly-ICLC with a desired range of molecular weights has been unavailable.

### BRIEF DESCRIPTION OF THE FIGURES

FIG. 1 is a table showing approximate Molecular weights (as determined by gel electrophoresis) in kilobases of Poly I and Poly C that was tested in the experiment detailed in preclinical Example 1. L=low, M=mid, H=high, as defined by the kilobase ranges shown.

FIG. 2 presents dose titration curves IFN-I production responses of various poly-ICLC preparations made with different molecular weight poly-I and poly-C homopolymers used in the experiment detailed in preclinical Example 1.

FIG. 3 is a chart showing IFN-I-production by MDA5 reporter cell line induced by various preparations of poly-ICLC at a concentration of 3.3 ng/ml., in the experiment detailed in preclinical Example 1.

FIG. 4 is a chart showing the Interferon response to various preparations of Poly-ICLC at a concentration of 100 ng/ml, as driven by MDA5 activation in RIG-1 knockout reporter cells in the mutant cell experiment detailed in preclinical Example 2; in that figure, ‘Dalt’ and ‘B03’ are separate historical poly-ICLC preparations. RLU ratio=relative light units.

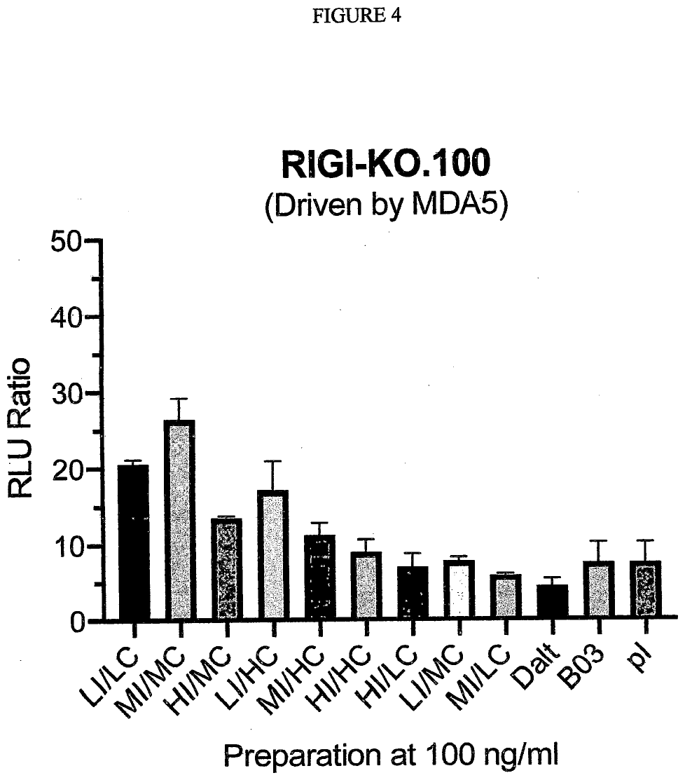

### SUMMARY OF THE INVENTION

The invention refers to new Poly-ICLC molecules and methods for producing them, and demonstrates that certain specific molecular weight molecules of the poly-I and poly-C components result in significantly improved activity of the final compound in certain applications. Specific examples include a Poly-ICLC molecule comprising a poly-I component which is approximately 0.4 to 5 Kilobases long and a poly-C component which is approximately 2 to 8 kilobases long, with specific benefits shown for subranges wherein the poly-I component is 2 to 4 Kilobases long and the poly-C component is 3 to 5 kilobases long and wherein the poly-C component is 2-4 Kilobases long and the poly-I component is 4-5 kilobases long.

These molecules may be incorporated in pharmaceutically or veterinary acceptable excipients and carriers for a number of uses in humans, in domestic animals and in wild animals. Such uses include (but are not limited to) prophylaxis pre-exposure prophylaxis, treatment and/or inflammatory symptom attenuation of viral or microbial infections; immunomodulating, vaccine adjuvant, antiviral, and/or anti-inflammatory effects mediated through activation of the MDA5, TLR3 and other dsRNA dependent enzyme systems; antineoplastic effects either alone or when combined with therapeutic vaccine or other anticancer immunologic agents; preventive cancer vaccine adjuvant effects in patients at risk for cancer. The molecules may be of particular use against SARS-CoV-2 infection or a cytokine storm caused by a SARS-CoV-2 infection. While dosage may be adjusted depending on the specific target and the specific patient, the dose should be sufficient to activate the MDA5 and TLR3 enzyme systems in the patient. For humans the dose would be between 0.5 and 50 micrograms per kilogram body weight. The nature of the molecule does not demand any specific route of administration. Therefore, the route can be selected based on the specific target and the specific patient, and could include (but not be limited to) IM, SC, IT, IV or IN.

Preferably, administration comprises two or three repeated dose cycles spaced 24 to 96 hours apart. Depending on the target and the patient's response, dose cycles may be repeated 2 to 4 times per month.

The present invention relates to optimization of bioactivity of the Poly-ICLC molecule, and more specifically, the enhancement of dual activation of TLR3 and MDA5. The inventors have found that such optimization can be achieved by controlling the range of molecular weights of the polyribonucleotide components and certain elements of the formulation process. In another embodiment, the present invention refers to Poly-ICLC molecules having optimized biological activity, as well as techniques for producing such molecules, compositions comprising the molecules, methods of treatment and use thereof.

### DETAILED DESCRIPTION OF THE INVENTION EMBODIMENTS

In a first preferred embodiment, the present application refers to Poly-ICLC molecules that maximize activation of certain enzyme systems, particularly the dsRNA dependent MDA5 helicase, and that comprise a poly-I component which is 0.4-5 kilobases long and a poly-C component which is 2-8 kilobases long. The Poly-ICLC disclosed herein may preferably comprise a poly-I component that is 2-4 Kilobases long and a poly-C component that is 3-5 kilobases long, or a poly-C component which is 2-4 Kilobases long and a poly-I component which is 0.4-5 kilobases long 1. The Poly-ICLC molecules disclosed herein have been found to be useful in the prophylaxis, pre-exposure prophylaxis, treatment and/or inflammatory symptom attenuation of viral or microbial infections.

In another preferred embodiment, the present application refers to a Poly-ICLC composition comprising the Poly-ICLC molecules disclosed herein. Such composition may be either for human or animal use, hence comprise pharmaceutically or veterinary acceptable excipients and carriers.

The phrase “pharmaceutically or veterinarily acceptable” excipient, vehicle or carrier refers to molecular entities and compositions that do not produce adverse, allergic, or other untoward reactions when administered to an animal or a human. As used herein, “pharmaceutically acceptable carrier” includes all solvents, dispersion media, coatings, antibacterial and antifungal agents, isotonic and absorption delaying agents and the like. The use of such media and agents for pharmaceutically active substances is well known in the art. Supplementary active ingredients also can be incorporated into the compositions.

The active compositions of the present invention may include classic pharmaceutical preparations. Administration of these compositions according to the present invention will be via any common route so long as the target tissue is available via that route. Although, the intramuscular, intratumor, and nasal routes are a preferred embodiment, other routes of administration are contemplated. This includes subcutaneous, intradermal, oral, nasal, buccal, rectal, vaginal, or topical. Such compositions would normally be administered as pharmaceutically or veterinarily acceptable compositions.

Solutions of the active compound can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions. In most cases the form must be sterile and must be fluid to the extent that easy administration by a syringe is possible. It must be stable under the conditions of manufacture and storage. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, using a coating, such as lecithin, by the maintenance of the required particle size in the case of dispersion and using surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin. Sterile injectable solutions are prepared by incorporating the active compounds in the required amount in the appropriate solvent with various of the other ingredients enumerated above, as required. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are spray-drying, vacuum-drying and freeze-drying techniques which yield a powder of the active ingredient plus any additional desired ingredient from a previously sterile-filtered solution thereof.

The preparation of more, and/or highly, concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and/or in such amount as is therapeutically effective. The formulations are easily administered in a variety of dosage forms, such as the type of injectable solutions described above, but drug release capsules and/or the like can also be employed.

For parenteral administration in an aqueous solution, for example, the solution should be suitably buffered, for example with Phosphate-buffered saline (PBS), if necessary and/or the liquid diluent first rendered isotonic. These aqueous solutions are especially suitable for intravenous, intramuscular, subcutaneous and/or intraperitoneal administration. In this connection, sterile aqueous media which can be employed will be known to those of skill in the art considering the present disclosure. Some variation in dosage will necessarily occur depending on the condition of the subject being treated. The person responsible for administration will, in any event, determine the appropriate dose for the individual subject. For intranasal administration, the pharmaceutical compositions may be nasal sprays, nasal drops, metered dose inhalers (MDI), dry powder inhalers (DPI), solutions administered by nebulizers, insufflation powders or nasal powders and the like. The pharmaceutical compositions of the present invention may be administered by any suitable methods used for delivery of the drugs to the systemic circulation.

The various dosage forms according to the present invention may comprise pharmaceutically acceptable nasal carrier or excipients suitable for formulating the same. The composition according to the present invention may include excipients that are well known in the art, such as, acidifying agent, pH regulators, chelating agents, preservatives, thickening agents, co-solvents, permeation enhancers, diluents, mucoadhesives, absorption enhancing agents and vehicle. The carrier(s) or excipients must be “acceptable” in the sense of being compatible with the other ingredients of the formulation and not deleterious to the recipient thereof. Such carriers are well known to those skilled in the art of pharmacology.

Upon formulation, solutions will be administered in a manner compatible with the dosage formulation and in such amount as is therapeutically effective. The formulations are easily administered in a variety of dosage forms such as injectable solutions, intranasal compositions, drug release capsules and the like.

Poly-ICLC may be generally formulated as per the method of Levy '82 (U.S. Pat. No. 4,349,538). Once formulated, the poly-ICLC becomes a new molecule that cannot be separated into its initial components. The proportions of the principal components by weight are: one part poly 1, to one part poly C, to 1.5 parts poly L, to 5 parts CMC (carboxymethylcellulose). These proportions essentially remain the same within the poly-ICLC molecule regardless of dilutions or delivery vehicles.

The compositions disclosed herein may be used in the prophylaxis, pre-exposure prophylaxis, treatment and/or inflammatory symptom attenuation of viral or microbial infections, as they have immunomodulating, antiviral, and/or anti-inflammatory effects, which are mediated through activation of TLR3, MDA5 RIG-I and other dsRNA dependent enzyme systems. Given the benefit of this disclosure, those of ordinary skill in the art could select specific formulations and choice of concentrations (dose) and excipients for specific patients and conditions. For example, in a most preferred embodiment, the Poly-ICLC composition comprising optimized poly-ICLC should be administered to humans or animals in a dose sufficient to activate the MDA5 and TLR3 enzyme systems in said patient, for example between 0.5 and 50 micrograms per kilogram of body weight. The same poly-ICLC compositions disclosed herein can be administered directly intramuscularly (IM), subcutaneously (SC), intratumorally (IT), intravenously (IV) or intranasally (IN) in two or three repeated dose cycles spaced 24 to 96 hours apart. The dose cycles can be repeated 2 to 4 times per month, for example. These can be administered to patients either alone, as adjuvants coadministered or mixed in with vaccine antigen, or in combination with certain chemotherapies, targeted therapies or other specific immunomodulatory molecules such as immune checkpoint inhibitors commonly used in cancer patients and well described in the Art.

In various embodiments, depending on logistical delivery requirements, the Poly-ICLC composition described here can be administered in any of various concentrations containing from 0.2 to 2 mg Poly-IC per milliliter Poly-ICLC colloidal suspension in sufficient quantity to deliver the desired dose.

Preferred doses are between 0.1 mg to 2 mg of the active ingredient Poly-IC. An effective amount is approximately 1 mg of Poly-IC per dose. For veterinary uses, the effective amount can be a dose of around 0.3 mg of poly-IC, but it may vary depending on the weight and species of the animal.

### Preclinical Examples

1) Low, middle and high molecular weight homopolymers of poly-I and poly-C were manufactured per the method of Michels and Pearson PCT 63/103,487, 2021 as follows: “This is a scalable process for production of polyribonucleotides of controlled molecular weight range through variation of processing time and input concentrations. Key elements include a method for immobilization of polynucleotide phosphorylase which has been covalently attached to an amino-functionalized solid support via a glutaraldehyde linkage; a method of repeatedly reacting inosine diphosphate or cytidine diphosphate monomers with immobilized polynucleotide 10 phosphorylase to produce polyribonucleotide chains; control of the chain length of Poly (I) and Poly (C) by varying cofactor concentration and the length of reaction time; a method for controlled and efficient large-scale manufacture of a specific, determined range of molecular weight poly I and poly C homopolymer chains.”

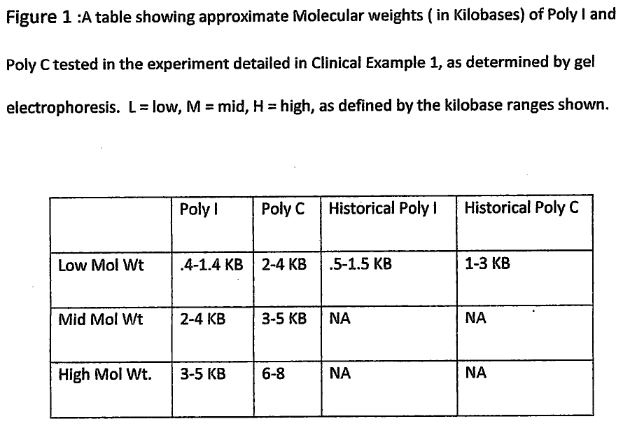

2) The average sizes in kilobases (kb) for each of the homopolymers are shown in FIG. 1 below. In addition, the average sizes of poly-I and poly-C of historical material which have been used for preparing historical poly-ICLC lots are shown.

FIG. 1: Approximate Molecular weights of Poly I and Poly C tested as determined by gel electrophoresis

| | | | | Historical | Historical |
|---|---|---|---|---|---|
| | Poly I | | Poly C | Poly I | Poly C |
| Low Mol Wt | .4-1.4 | KB | 2-4 KB | .5-1.5 KB | 1-3 KB |
| Mid Mol Wt | 2-4 | KB | 3-5 KB | NA | NA |
| High Mol Wt. | 3-5 | KB | 6-8 | NA | NA |

Nine preparations of poly-IC were made by mixing all possible combinations as follows: (LMW=low molecular weight, MMW=mid molecular weight, HMW=high molecular weight as detailed in FIG. 1.)
1. LMW poly-1+LMW poly-C=LI/LC
2. LMW poly-I+MMW poly-C=LI/MC
103. LMW poly-I+HMW poly-C=LI/HC
4. MMW poly-1+LMW poly-C=MI/LC
5. MMW poly-1+MMW poly-C=MI/MC
6. MMW poly-1+HMW poly-C=MI/HC
7. HMW poly-I+LMW poly-C=HI/LC
8. HMW poly-I+MMW poly-C=HI/MC
9. HMW poly-I+HMW poly-C=HI/HC
10. Historical (Dalton) poly-1+Historical (Dalton) poly-C=DI/DC

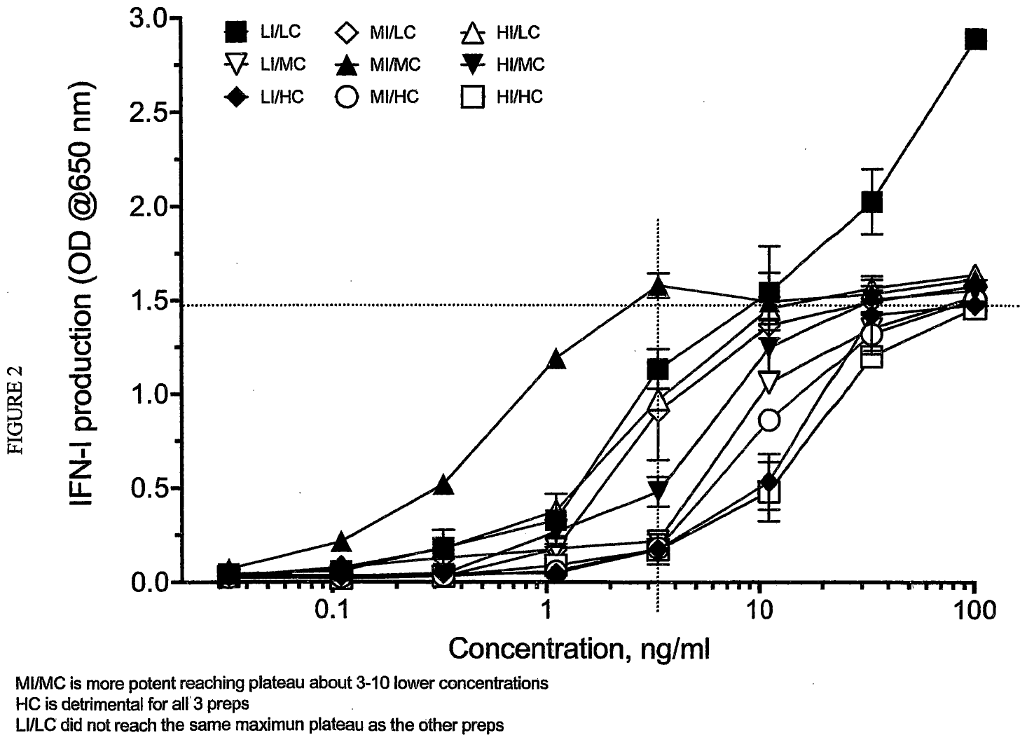

After preparing the poly-I/poly-C duplexes, these were combined with carboxymethyl cellulose (CMC) poly-Lysine to make 10 different preparations of poly-ICLC, using the Method of Levy '82 (U.S. Pat. No. 4,349,538, incorporated herein by reference). The 10 poly-ICLC preparations were tested at various concentrations for their ability to induce IFN-I production using a HEK-Blue human IFN reporter cell line, commercially available from Invivogen, specifically designed to measure MDA5 responses as described above (Invivogen, Inc.) Results of the dose titration curves are shown in FIG. 1. Comparison of responses using these preparations at a concentration of 3.3. ng/ml is shown in FIG. 2.

The results indicate a marked difference between preparations in activation of the MDA5 weighted bioassay: The MI/MC combination exhibited the highest potency as compared to the other preparations. Unexpectedly, all 3 preparations made with HC, which is the largest polynucleotide, had significantly lower activity as compared to the rest. The 3 preparations made with LC had similar intermediate activity and were comparable to historical lots of poly-ICLC.

3) PRECLINICAL EXAMPLE 2: Mutant Knockout Experiment.

In order to further confirm the relation of the invention to activation through MDA5, we also tested the same combinations listed above in Table 1, but this time using mutant MDAS and RIG-I knockout reporter cells that distinguish between activation of the two pathways.

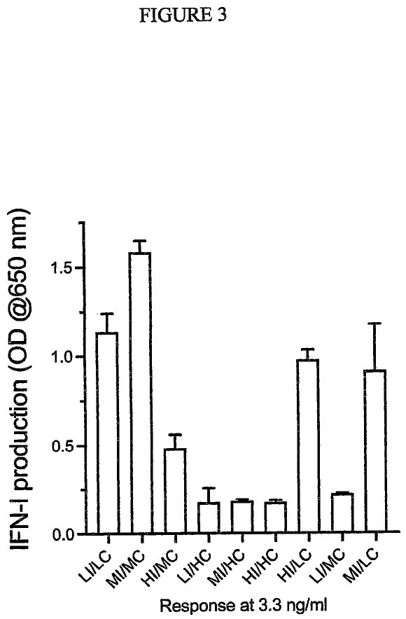

FIG. 3 again confirms the clear superiority of the MI/MC and LI/LC preparations in RIG-I knockout cells restricted to using the MDA5 pathway, with responses up to several times higher than historical Poly-ICLC (Dalt. and B03) as well as other preparations. Results in FIG. 3 are in concordance with those in FIG. 2 using wild-type cells, showing the MI/MC preparation to be superior. In addition, not shown are the results of the MDAS mutant RIG-I knockout experiment at a higher dose of 300 ng, which actually show less difference in MDAS activation between preparations, and thus implying a dose response. This suggests that lower (‘stress’) doses of Poly-ICLC formulated as disclosed here and in the claims may be better if the target is MDA5. The important clinical implications of these findings are discussed in the background section above, but given the criticality of the MDA5 pathway to the multiple steps in the immune response to Poly-ICLC, they could include marked improvement in responses to cancer, viral, and inflammatory indications. This is illustrated in the cancer autovaccination strategy described under embodiments above and in preclinical studies. (Sultan, Salazar, Celis, September 2020)

Based on these results in both wild type and mutant cells, we conclude that the Mid Poly-I/Mid Poly-C combination as shown in table FIG. 1 will produce the optimal formulation of poly-ICLC for generation of clinical antitumor, antiviral and anti-inflammatory effect via both TLR3 and especially MDA5. Formulations using LI/LC will be slightly less effective but still adequate, whereas other formulations will be significantly less active.

One of ordinary skill in the art could further characterize the compounds precisely using size exclusion chromatography combined with multi-angle light scattering (SEC-Mals); SEC-UV, or other tools well known in the art particularly for determining molecular weight of the Poly I and poly C homopolymer components as well as of the IC portion of the final Poly-ICLC molecule.

Similarly, one of ordinary skill in the art could determine the level of TLR3 activation by the various compounds by using a readily available commercial TLR3 assay. (Invivogen Inc.) Finally, one of ordinary skill in the art could confirm the in-vivo and clinical activity of the different compounds, including with regard to their activity in mice that are TLR3 or MDA5 deficient, as described by Sultan, Celis et al 2018.

Below, some clinical trial examples are given for the sake of clarity and sufficient disclosure

### Clinical Example 1, Cancer

In one particular clinical example, Poly-ICLC can be administered to cancer patients in a comprehensive therapeutic ‘Autovaccination’ strategy that converts the tumor into its own personalized therapeutic vaccine and then maintains and facilitates its action. This method consists of intratumoral (IT) injection of 0.5 to two mg Poly-ICLC to induce cancer cell apoptosis, release tumor antigens and activate dendritic cells (DC) via TLR3, thus inducing an immune prime including T-cells against these antigens. This is followed by IM administration of 1 to two mg Poly-ICLC to expand those specific anti-tumor killer T-cells via MDA5 and Interleukin-15, as well as to target and facilitate infiltration of those cells into tumor via MDA5, interferon, and cytokine CXCL-10 through a direct effect on tumor endothelium. Preliminary results of this strategy have proven the concept in patients with various solid cancers, including sarcoma, lymphoma, and prostate cancer. (Salazar et al 2014; Kyi et al 2018; Sultan, Salazar, Celis, September 2020) An additional step comprise preconditioning of the immune response by reducing myeloid derived suppressor cells (MDSC) via MDA5 through administration of one to two mg Poly-ICLC two to three times per week for 1-2 weeks prior to the intratumoral ‘autovaccination’, or vaccine administration as the case may be.

As can be seen from this clinical example, optimal activation of MDAS is critical to the success of this strategy, which is working at multiple steps of the recently clarified sequential immune response to cancer discussed in the background above. (Sultan, Salazar, Celis, September 2020). This same concept applies to therapeutic cancer vaccination using exogenous antigens or T-cell therapies.

### Clinical Example 2, Viral Prophylaxis, COVID-19

In another Clinical example, optimized Poly-ICLC per this disclosure can be administered intranasally to patients for prevention and/or attenuation of COVID-19, and the cytokine storm in other viral respiratory infections. Poly-ICLC could be administered in a cycle consisting of a dose of 0.5 to 1 mg per nostril on day 1 followed by repeat dosing 48 to 72 hours later. This can be delivered as a liquid nasal spray, by instillation, or in powdered form.

In preclinical challenge models of SARs CoV-1, SARS COV-2 and influenza, such treatment administered beginning 1 to 14 days prior to lethal viral challenge prevented infection and/or completely protected mice from death. Some animals that exhibited low viral titres developed robust immunity to reinfection. (Kumaki, Salazar, Barnard 2012) In other words, nasal poly-ICLC converted the infection into an attenuated live virus vaccine equivalent. This protection appears to have been conferred by both an antiviral effect and an MDA5-mediated antiinflammatory effect as discussed above in the background to this disclosure, thus again emphasizing the importance of optimizing MDA5 activation. Because of the importance of the MDA5-mediated antiinflamatory effect to this protection, the new Poly-ICLC compounds claimed herein are expected to have a much improved protective effect.

As Illustrated, the present application also refers to medical and veterinary uses of the poly-ICLC molecules or compositions disclosed herein. In this specific embodiment, the use is for preparing a medicament for pre-exposure prophylaxis, treatment and/or inflammatory symptom attenuation of viral or microbial infections via intranasal administration.

### REFERENCES

Additional information is contained in the following publicly available materials:
Caskey, M., F. Lefebvre, . . . , A. Salazar, S. Schlesinger, R. Steinman and R. Sekaly, et al (2011). “Synthetic double stranded RNA reliably induces innate immunity similar to a live viral vaccine in humans” J Exp Med 208.
Kato H. Takeuchi O . . . Akira S, et al, (2008)” Length dependent recognition of dsRNA by Rigl and MDA5. JEM 205 (7) 1601-1610
Morahan P, et al (1972) Antiviral activity and side effects of Poly IC complexes as affected by molecular size. PNAS 69 (4) 842-46.
Gesuete R, Christensen S, Bahjat F, PhD, 1 Amy E. B. Packard A . . . , Salazar A M, Stenzel Poore M. et al. (2016). Cytosolic receptor MDA5 mediates Poly-ICLC preconditioning induced neuroprotection against cerebral ischemic injury. Stroke. 2016 January; 47 (1): 262-6. doi: 10.1161/STROKEAHA.115.010329. Epub 2015 Nov. 12. PMID: 265641
Sultan H, Wu J, Kumai T, Salazar A M, Celis E. (2018) Role of MDA5 and interferon-I in dendritic cells for T cell expansion by anti-tumor peptide vaccines in mice. (2017) Cancer Immunol Immunotherapy. https://doi.org/10.1007/s00262-018-2164-6
Sultan H, Salazar AM, and Celis, E (October 2020) Poly-ICLC, a multifunctional immune modulator for treating cancer. Seminars in Immunology, Review, https://doi.org/10.1016/j.smim.2020.101414
Kumaki, Y., A. M. Salazar, M. K. Wandersee and D. L. Barnard (2017). “Prophylactic and therapeutic intranasal administration with an immunomodulator, Hiltonol ((R)) (Poly IC: LC), in a lethal SARS COV-1 infected BALB/c mouse model.” Antiviral Res 139:1-12.
Salazar, A M, et al (2014) Salazar, A. M., Erlich, R. B., Mark, A., Bhardwaj, N. & Herberman, R. B. Therapeutic in situ autovaccination against solid cancers with intratumoral poly-ICLC: case report, hypothesis, and clinical trial. Cancer Immunol Res 2, 720-724, doi: 10.1158/2326-6066.CIR-14-0024 (2014).
Kyi, C. et al. Therapeutic Immune Modulation against Solid Cancers with Intratumoral Poly-ICLC: A Pilot Trial. Clin Cancer Res 24, 4937-4948, doi: 10.1158/1078-0432. Ccr-17-1866 (2018).

What is claimed is:

1. A Poly-ICLC molecule comprising a poly-I component which is approximately 0.4 to 5 Kilobases long and a poly-C component which is approximately 2 to 8 kilobases long.

2. The Poly-ICLC molecule of claim 1, wherein the poly-I component is approximately 2 to 4 Kilobases long and the poly-C component is approximately 3 to 5 kilobases long.

3. The Poly-ICLC molecule of claim 1, wherein the poly-C component is approximately 2-4 Kilobases long and the poly-I component is approximately 4-5 kilobases long.

4. A Poly-ICLC composition comprising the Poly-ICLC molecule of any one of claim 1-3 and pharmaceutically or veterinary acceptable excipients and carriers for delivery in liquid or powder form.

5. The Poly-ICLC molecule of claims 1-3, or composition of claim 4, for use in the prophylaxis, pre-exposure prophylaxis, treatment and/or inflammatory symptom attenuation of viral or microbial infections.

6. The Poly-ICLC composition of claim 4, wherein the composition has immunomodulating, vaccine adjuvant, antiviral, and/or anti-inflammatory effects mediated through activation of the MDA5, TLR3 and other dsRNA dependent enzyme systems.

7. The Poly-ICLC composition of claim 4 wherein the composition has multiple antineoplastic effects either alone or when combined with therapeutic vaccine or other anticancer immunologic agents such as immune checkpoint inhibitors.

8. The Poly-ICLC composition of claim 4, wherein the composition has preventive cancer vaccine adjuvant effects in patients at risk for cancer.

9. The Poly-ICLC composition of claim 4 wherein the dose of optimized poly-ICLC administered to human is between 0.5 and 50 micrograms per kilogram body weight, sufficient to activate the MDA5 and TLR3 enzyme systems in said patient

10. The composition of claim 4 wherein the dose of optimized Poly-ICLC administered to an animal is between 0.5 and 50 micrograms per kilogram body weight, sufficient to activate the MDA5 and TLR3 systems in said animals

11. The composition of claim 10 wherein said animal is a domestic animal.

12. The composition of claim 10 wherein said animal is a wild animal

13. The Poly-ICLC composition of claim 4 wherein the poly-ICLC is administered IM, SC, IT, IV or IN in two or three repeated dose cycles spaced 24 to 96 hours apart.

14. The Poly-ICLC composition of claim 7 wherein the dose cycles are repeated 2 to 4 times per month.

15. Use of the Poly-ICLC molecule of claims 1-3 or composition of claim 4, wherein the use is for preparing a medicament for pre-exposure prophylaxis, treatment and/or inflammatory symptom attenuation of viral or microbial infections.

16. Use, according to claim 10, wherein the medicament is for intranasal administration in either liquid or powder form.

17. Use, according to any one of claim 10 or 11, wherein the viral infection is a SARS-CoV-2 infection.

18. Use, according to claim 10, wherein the inflammatory symptom is a cytokine storm caused by a SARS-CoV-2 infection.
